(19) United States
(12) Patent Application Publication
(10) Pub. No.: US 2024/0225458 A9
(43) Pub. Date: Jul. 11, 2024

(54) CUFFLESS BLOOD PRESSURE ESTIMATING DEVICE USING HYDROSTATIC PRESSURE DIFFERENCE AND OPERATING METHOD THEREOF

(71) Applicant: ELECTRONICS AND TELECOMMUNICATIONS RESEARCH INSTITUTE, Daejeon (KR)

(72) Inventors: Chanki PARK, Daejeon (KR); Hyun Soon SHIN, Daejeon (KR)

(21) Appl. No.: 18/491,921

(22) Filed: Oct. 23, 2023

(30) Foreign Application Priority Data
Oct. 24, 2022 (KR) 10-2022-0137684

(51) Int. Cl. A61B 5/021; A61B 5/02; A61B 5/318

(52) U.S. Cl. CPC A61B 5/021; A61B 5/02007; A61B 5/318; A61B 5/02416

(57) ABSTRACT

Disclosed is a cuffless blood pressure estimating device, which includes a hemodynamic parameter estimating circuit that measures at least two height levels based on position information output from at least one position detection sensor, measures user's bio-signals respectively at the height levels, and estimates a blood pressure from the height levels and the user's bio-signals based on a machine learning algorithm or a state estimation algorithm with a hemodynamic state space model, and at least one processor that controls the hemodynamic parameter estimating circuit.

### CROSS-REFERENCE TO RELATED APPLICATION

This application claims priority under 35 U.S.C. § 119 to Korean Patent Application No. 10-2022-0137684, filed on Oct. 24, 2022, in the Korean Intellectual Property Office, the disclosures of which are incorporated by reference herein in their entireties.

### BACKGROUND

### 1. Field of the Invention

Embodiments of the present disclosure described herein relate to a cuffless blood pressure estimating device using a hydrostatic pressure difference and operating method thereof, and more particularly, relate to a cuffless blood pressure estimating device using a hydrostatic pressure difference based on user's bio-signals measured from at least two heights and operating method thereof.

### 2. Description of Related Art

As smart devices develop, the healthcare-related sensing device market is growing. Accordingly, various blood pressure estimating devices are being developed.

The blood pressure estimating devices are classified into an invasive method in which a needle is inserted into a blood vessel and a non-invasive method in which external measurement is performed. The non-invasive method is divided into a cuff method using a cuff actuator and a cuffless method. The cuffless method is easy to measure, but has issues of low accuracy. To solve the issues, conventional cuffless blood pressure estimating devices need to be calibrated regularly in conjunction with a cuff-type external blood pressure monitor.

### SUMMARY

Embodiments of the present disclosure provide a cuffless blood pressure estimating device and operating method thereof with a calibration-free function or a minimum calibration function using a hydrostatic pressure difference.

According to an embodiment of the present disclosure, a cuffless blood pressure estimating device includes a hemodynamic parameter estimating circuit that measures at least two height levels based on position information output from at least one position detection sensor, measures user's bio-signals respectively at the height levels, and estimates a blood pressure from the height levels and the user's bio-signals based on a machine learning algorithm or a state estimation algorithm with a hemodynamic state space model; and at least one processor that controls the hemodynamic parameter estimating circuit.

According to an embodiment of the present disclosure, a method of operating a cuffless blood pressure estimating device for estimating a blood pressure includes measuring at least two height levels based on position information output from at least one position detection sensor, measuring user's bio-signals respectively at the height levels, estimating a blood pressure from the height levels and the user's bio-signals based on a machine learning algorithm or a state estimation algorithm with a hemodynamic state space model.

### BRIEF DESCRIPTION OF THE DRAWINGS

A detailed description of each drawing is provided to facilitate a more thorough understanding of the drawings referenced in the detailed description of the present disclosure.

FIG. 1 illustrates a change in a blood pressure according to a distance from a heart as an example.

FIG. 2 is a block diagram illustrating a cuffless blood pressure estimating device, according to an embodiment of the present disclosure.

FIG. 3 is a block diagram illustrating a hemodynamic parameter estimating circuit of FIG. 2 as an example.

FIG. 4 illustrates an operation of a cuffless blood pressure estimating device as an example, according to an embodiment of the present disclosure.

FIGS. 5A and 5B illustrate methods for determining a reference level of a hemodynamic parameter estimating circuit as examples, according to an embodiment of the present disclosure.

FIG. 6 is a block diagram illustrating a cuffless blood pressure estimating device, according to an embodiment of the present disclosure.

### DETAILED DESCRIPTION

Hereinafter, embodiments of the present disclosure may be described in detail and clearly to such an extent that an ordinary one in the art easily implements the present disclosure.

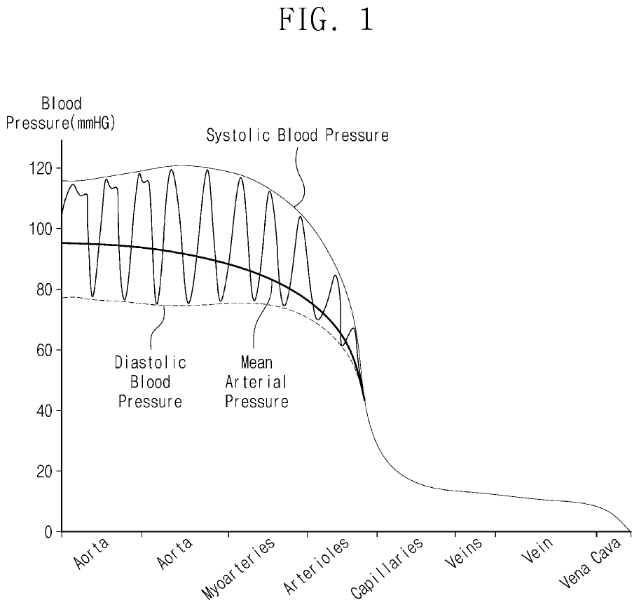

FIG. 1 illustrates a change in a blood pressure according to a distance from a heart as an example. Referring to FIG. 1, the blood pressure corresponding to a systolic phase of the heartbeat is referred to as a systolic blood pressure, the blood pressure corresponding to a diastolic phase of the heartbeat is referred to as a diastolic blood pressure, and the averaged blood pressure is referred to as a mean arterial pressure. The blood pressure measured in each blood vessel may drop compared to the blood pressure in an aorta, but it is usually assumed that there are no significant differences in blood pressures within large arteries.

When measuring blood pressure from the forearm, wrist, and finger, it should be considered whether the height level of the sensor is the same as the heart level. When the height level of the sensor and the heart level do not match, a difference in hydrostatic pressure may occur, and the measured blood pressure may be biased. For example, ‘measured blood pressure in the hand (radial artery)=actual blood pressure−hydrostatic pressure in the hand (radial artery)’. In this case, the hydrostatic pressure may be ‘gravity acceleration*blood density*difference between the height level of the sensor and the heart level’. According to an embodiment, the blood pressure may be estimated based on the hydrostatic pressure.

For cuffless and calibration-free (or minimum calibration) blood pressure estimation, a photoplethysmogram (PPG) signal, an electrocardiogram (ECG) signal, or a pulse transit time (PTT) value may be required. In this case, the PPG signal may be a signal obtained by measuring the amount of the change in blood volume through an optical sensor, the ECG signal may represent the difference in electrical potential according to heartbeat, and the PTT value may be the time lag between the systolic upstroke timing of each heartbeat and that of the corresponding pulse at the measurement site. The PTT value may be a value calculated from the PPG signal and the ECG signal.

Since the PPG signal represents the amount of change in blood volume, a change in blood vessel characteristics may be estimated from the waveform of the PPG signal. Accordingly, the waveform of the PPG signal may contain information about the blood pressure. The PPG signal may vary depending on the height level at which it is measured.

The PPG signal (or the waveform of the PPG signal) and the PTT value may depend on hemodynamic parameters (or hemodynamic states) such as blood pressure, blood density, elastic modulus, vessel wall thickness, vessel radius, and vessel length, and the like. The cuffless blood pressure estimation device may be calibrated to compensate for the effect of these hemodynamic parameters except for the blood pressure using an external cuff type blood pressure monitor.

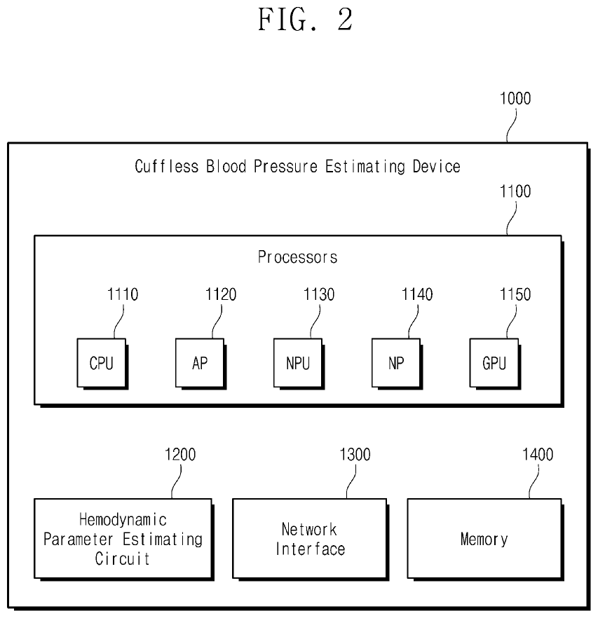

FIG. 2 is a block diagram illustrating a cuffless blood pressure estimating device 1000, according to an embodiment of the present disclosure. Referring to FIG. 2, the cuffless blood pressure estimating device 1000 may include processors 1100, a hemodynamic parameter estimating circuit 1200, a network interface 1300, and a memory 1400.

The processors 1100 may perform functions as a central processing unit of the blood pressure estimating device 1000. At least one of the processors 1100 may include at least one general-purpose processor such as a central processing unit 1110 (CPU) or an application processor 1120 (AP). The processors 1100 may also include at least one special purpose processor, such as a neural processing unit 1130, a neuromorphic processor 1140, a graphics processing unit 1150 (GPU), etc. The processors 1100 may include two or more homogeneous processors. As another example, at least one of the processors 1100 may be manufactured to implement various machine learning or deep learning modules. For example, at least one of the processors 1100 may perform machine learning to determine a regression model as a hemodynamic state space model.

At least one of the processors 1100 may control the hemodynamic parameter estimating circuit 1200. At least one of the processors 1100 may control the hemodynamic parameter estimating circuit 1200 to measure at least two height levels based on position information output from at least one position detection sensor. For example, at least one of the processors 1100 may control the hemodynamic parameter estimating circuit 1200 to measure a first height level higher than or equal to a reference level and a second height level lower than or equal to the reference level.

At least one of the processors 1100 may control the hemodynamic parameter estimating circuit 1200 to determine the reference level to estimate the blood pressure. The reference level may be a value representing a height level from the ground to the user's heart. For example, at least one of the processors 1100 may control the hemodynamic parameter estimating circuit 1200 to determine the reference level based on heart height level information of the user. At least one of the processors 1100 may control the hemodynamic parameter estimating circuit 1200 to determine the reference level from the first height level and the second height level. At least one of the processors 1100 may control the hemodynamic parameter estimating circuit 1200 to determine the reference level from a trajectory of the user's hand.

At least one of the processors 1100 may control the hemodynamic parameter estimating circuit 1200 to measure user's bio-signals respectively at the height levels. In this case, the user's bio-signals may be the PPG signals or the ECG signals. For example, at least one of the processors 1100 may control the hemodynamic parameter estimating circuit 1200 to measure a first PPG signal at the first height level and a second PPG signal at the second height level.

At least one of the processors 1100 may control the hemodynamic parameter estimating circuit 1200 to estimate the hemodynamic parameters (or hemodynamic states) from the height levels and the user's bio-signals based on a machine learning algorithm or state estimation algorithm with a hemodynamic state space model. The hemodynamic parameters may include information on one or more of a blood pressure, a blood density, a blood vessel elastic modulus, a vessel wall thickness, a vessel radius, and/or a vessel length.

For example, the hemodynamic state space model may be determined based on Equations 1 to 5 below.

X(m)=[Pa, ρ, E, T(m), L]^{T} [Equation 1]

X(m)=f(X(m−1), H(m)) [Equation 2]

Z(m)=[PPG(m), PTT(m)]^{T} [Equation 3]

PPG(m)=g1(X(m), H(m)) [Equation 4]

PTT(m)=g2(X(m), H(m)) [Equation 5]

Here, X(m) represents a hemodynamic parameter vector (‘m’ is an index of height level of the position detection), Z(m) represents a feature vector having a size of k×1 (‘k’ is the number of measured features), Pa represents a blood pressure, ρ represents a blood density, ‘E’ represents an elastic modulus of blood vessels, T(m) represents a thickness of a vessel wall, R(m) represents a vessel radius, ‘L’ represents a vessel length, H(m) represents a difference value between a reference level and a m-th height level of the position detection sensor (e.g., H(1) is a first difference value, which is a difference between the reference level and the first height level of the position detection sensor, and H(2) is a second difference value, which is a difference between the reference level and the second height level of the position detection sensor), PPG(m) represents a feature vector of a PPG signal measured at the m-th height level of the position detection sensor, PTT(m) represents a PTT value measured at the m-th height level of the sensor, and f( ) and g( ) represent functions based on hemodynamic formulas or mathematical models trained by the machine learning algorithm. In this case, one of the hemodynamic formulas may be Equation 6 below.

$$PTT = L \sqrt{\frac{2 B ρ}{LT}} \qquad [ Equation 6 ]$$

For example, g2(X(m)) may be expressed as Equation 7 below.

$$g 2 ( X ( m ) ) = L \sqrt{\frac{2 \cdot R ( m ) \cdot ρ}{E \cdot T ( m )}} \qquad [ Equation 7 ]$$

For example, when the user's bio-signals are measured at M positions and k features are extracted from each user bio-signals, k×M equations may be obtained. Therefore, it is possible to estimate the hemodynamic parameters having k×M or less independent variables. That is, it is possible to perform calibration-free or minimally calibrated cuffless blood pressure estimation. The number of independent variables may increase or decrease depending on the degree of detail or simplification of the hemodynamic state space model. When the number of positions to be measured increases, accuracy of hemodynamic parameter estimation increases, but processing time will be longer. When the number of positions to be measured decreases, accuracy of hemodynamic parameter estimation decreases, but processing time will be shorter.

At least one of the processors 1100 may control the hemodynamic parameter estimating circuit 1200 to extract information about the blood pressure from the hemodynamic parameters.

The hemodynamic parameter estimating circuit 1200 may measure at least two height levels. For example, the hemodynamic parameter estimating circuit 1200 may measure at least two height levels based on position information output from at least one position detection sensor under the control of at least one of the processors 1100. For example, the hemodynamic parameter estimating circuit 1200 may measure a first height level higher than or equal to the reference level and a second height level lower than or equal to the reference level under the control of at least one of the processors 1100.

The hemodynamic parameter estimating circuit 1200 may determine the reference level based on the user's heart height level information under the control of at least one of the processors 1100. The hemodynamic parameter estimating circuit 1200 may determine the reference level from the first height level and the second height level under the control of at least one of the processors 1100. The hemodynamic parameter estimating circuit 1200 may determine the reference level from a trajectory of the user's hand under the control of at least one of the processors 1100.

The hemodynamic parameter estimating circuit 1200 may measure the user's bio-signals respectively at the height levels under the control of at least one of the processers 1100. In this case, the user's bio-signals may be the PPG signal, the PTT, or the ECG signal. For example, the hemodynamic parameter estimating circuit 1200 may measure the first PPG signal at the first height level and the second PPG signal at the second height level under the control of at least one of processors 1100.

The hemodynamic parameter estimating circuit 1200 may estimate the hemodynamic parameters from the height levels and the user's bio-signals based on the machine learning algorithm or the state estimation algorithm with the hemodynamic state space model. The hemodynamic parameters may include information on one or more of a blood pressure, a blood density, a blood vessel elastic modulus, a vessel wall thickness, a vessel radius, and/or a vessel length.

For example, the hemodynamic parameter estimating circuit 1200 may extract a plurality of features from each of the user's bio signals. The hemodynamic parameter estimating circuit 1200 may estimate the hemodynamic parameters from the height levels and the plurality of the features based on the machine learning algorithm.

For example, the hemodynamic parameter estimating circuit 1200 may extract the plurality of the features from each of the user's bio signals. The hemodynamic parameter estimating circuit 1200 may estimate the hemodynamic parameters from the height levels and the plurality of the features based on the state estimation algorithm with the hemodynamic state space model. The state estimation algorithm may be one of an extended Kalman filter, an unscented Kalman filter, and a particle filter.

The hemodynamic parameter estimating circuit 1200 may extract information about the blood pressure from the hemodynamic parameters. For example, the hemodynamic parameter estimating circuit 1200 may extract information about the blood pressure from the hemodynamic parameters under the control of at least one of the processors 1100.

The network interface 1300 may provide remote communication with an external device. The network interface 1300 may perform wireless or wired communication with an external device. The network interface 1300 may communicate with an external device through at least one of various communication types such as Ethernet, Wi-Fi, LTE, and 5G mobile communication. For example, the network interface 1300 may communicate with an external device with respect to the cuffless blood pressure estimating device 1000.

The network interface 1300 may receive operation data to be processed by the cuffless blood pressure estimating device 1000 from an external device. The network interface 1300 may output result data generated by the cuffless blood pressure estimating device 1000 to an external device.

The memory 1400 may store data and process codes being processed or to be processed by the processors 1100. For example, in some embodiments, the memory 1400 may store data to be input to the cuffless blood pressure estimating device 1000 or data generated or trained in a process of performing deep learning by the processors 1100.

The memory 1400 may be used as a main memory device of the cuffless blood pressure estimating device 1000. The memory 1400 may include a dynamic random access memory (DRAM), a static RAM (SRAM), a phase-change RAM (PRAM), a magnetic RAM (MRAM), a ferroelectric RAM (FeRAM), a resistive RAM (RRAM), etc.

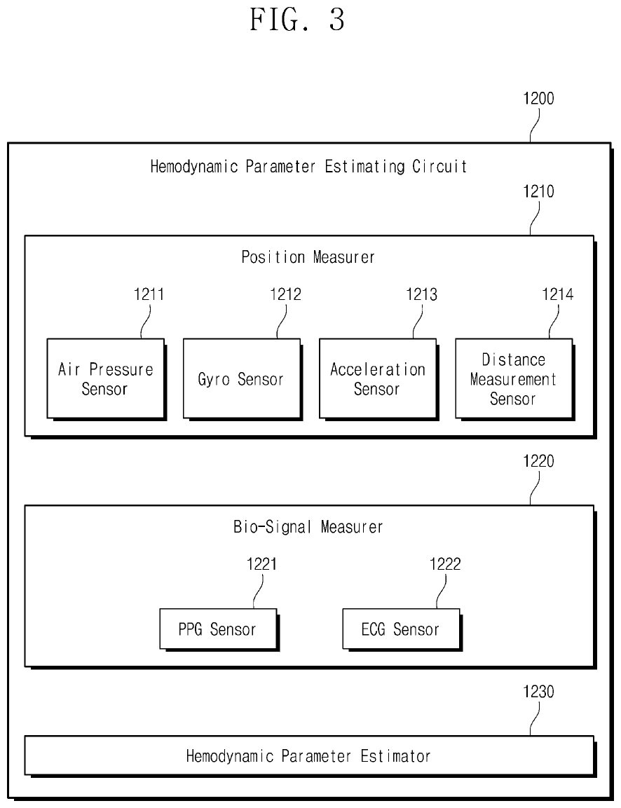

FIG. 3 is a block diagram illustrating the hemodynamic parameter estimating circuit 1200 of FIG. 2 as an example. Referring to FIGS. 2 and 3, the hemodynamic parameter estimating circuit 1200 may include a position measurer 1210, a bio-signal measurer 1220, and a hemodynamic parameter estimator 1230.

The position measurer 1210 may include at least one position detection sensor. For example, the position measurer 1210 may include an air pressure sensor 1211, a gyro sensor 1212 (e.g, a gyroscope), an acceleration sensor 1213 (e.g, an accelerometer), and a distance measurement sensor 1214. The air pressure sensor 1211 may be at least one of a barometer, altimeter, variometer. The distance measurement sensor 1214 may measure a distance using ultrasound or laser. Although not shown in FIG. 3, the position measurer 1210 may include a magnetometer.

The position measurer 1210 may measure at least two height levels based on position information output from at least one position detection sensor. For example, the position measurer 1210 may measure a first height level higher than or equal to a reference level and a second height level lower than or equal to the reference level based on position information output from at least one position detection sensor.

The position measurer 1210 may determine the reference level based on the user's heart height level information. The position measurer 1210 may determine the reference level from the first height level and the second height level. The position measurer 1210 may determine the reference level from the trajectory of the user's hand.

The position measurer 1210 may be a smart device including sensors. For example, the position measurer 1210 may be a smart phone, a smart watch, or a smart ring.

The bio-signal measurer 1212 may measure user's bio-signals respectively at height levels of a measurement site (e.g., a user's finger or wrist). In this case, the user's bio-signals may each include at least one of the PPG signal and the ECG signal. For example, the bio-signal measurer 1212 may measure a first PPG signal at the first height level and a second PPG signal at the second height level.

The bio-signal measurer 1212 may include a PPG sensor 1221 and an ECG sensor 1222. The PPG sensor 1221 may measure the PPG signal by measuring a change in blood volume of the one hand of the user. In this case, the PPG sensor may include an LED and a photo detector. The ECG sensor 1222 may measure the ECG signal by measuring electrical signals according to heartbeats from both hands of the user.

The bio-signal measurer 1212 may measure the PTT value from a time lag between the systolic upstroke timing of each heartbeat and that of the corresponding pulse at the measurement site (e.g., the user's finger or wrist). The bio-signal measurer 1212 may calculate the PTT value from the PPG signal and the ECG signal.

The bio-signal measurer 1212 may be a smart device including sensors. For example, the bio-signal measurer 1212 may be a smart phone, a smart watch, or a smart ring.

The hemodynamic parameter estimator 1230 may estimate the hemodynamic parameters from height levels and the user's bio-signals based on the machine learning algorithm or the state estimating algorithm with the hemodynamic state space model. The hemodynamic parameters may include information on one or more of a blood pressure, a blood density, a blood vessel elastic modulus, a vessel wall thickness, a vessel radius, and/or a vessel length.

For example, the hemodynamic parameter estimator 1230 may extract the plurality of the features from each of the user's bio signals. The hemodynamic parameter estimator 1230 may estimate the hemodynamic parameters from the height levels and the plurality of the features based on the machine learning algorithm.

For example, the hemodynamic parameter estimator 1230 may extract the plurality of the features from each of the user's bio signals. The hemodynamic parameter estimator 1230 may estimate the hemodynamic parameters from the height levels and the plurality of the features based on the state estimation algorithm with the hemodynamic state space model. The state estimation algorithm may be one of an extended Kalman filter, an unscented Kalman filter, and a particle filter.

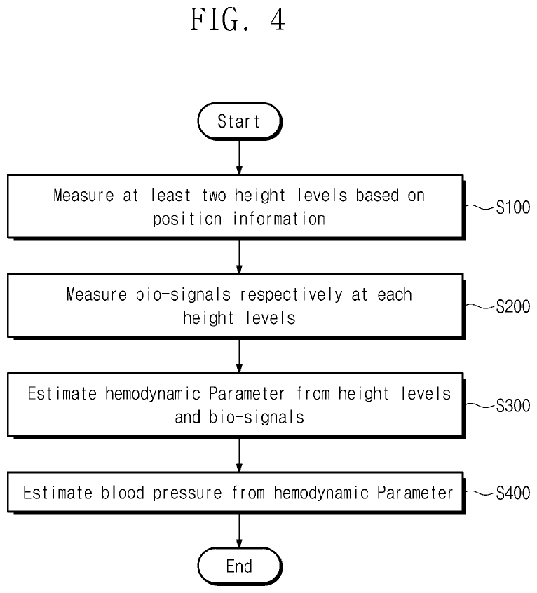

FIG. 4 illustrates an operation of the cuffless blood pressure estimating device 1000 as an example, according to an embodiment of the present disclosure. Referring to FIGS. 1 to 4, in operation S100, the hemodynamic parameter estimating circuit 1200 may measure at least two height levels based on position information output from at least one position detection sensor. In this case, the at least two height levels may include a first height level higher than or equal to the reference level and a second height level lower than or equal to the reference level.

In operation S200, the hemodynamic parameter estimating circuit 1200 may measure user's bio-signals respectively at each height level. In this case, the user's bio-signals may each include at least one of the PPG signal or the ECG signal.

In operation S300, the hemodynamic parameter estimating circuit 1200 may estimate the hemodynamic parameters from the height levels and the user's bio-signals based on the machine learning algorithm or the state estimation algorithm with the hemodynamic state space model. The hemodynamic parameters may include information on one or more of a blood pressure, a blood density, a blood vessel elastic modulus, a vessel wall thickness, a vessel radius, and/or a vessel length.

In operation S400, at least one of the processors 1100 may control the hemodynamic parameter estimating circuit 1200 to extract information about the blood pressure from the hemodynamic parameters.

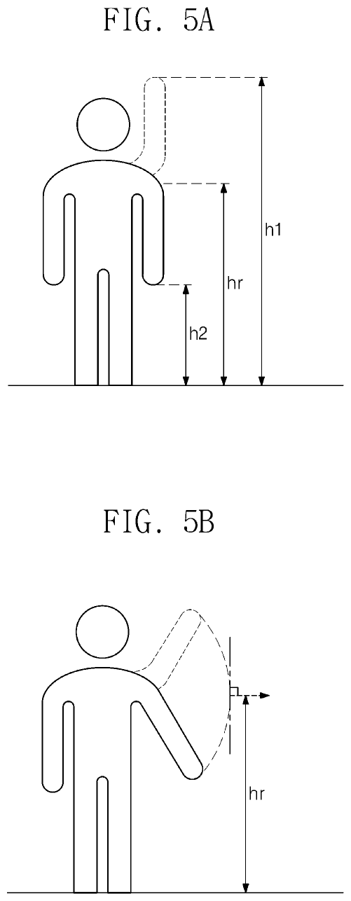

FIGS. 5A and 5B illustrate methods for determining a reference level of the hemodynamic parameter estimating circuit 1200 as examples, according to an embodiment of the present disclosure. Referring to FIGS. 2, 3, and 5A, the hemodynamic parameter estimating circuit 1200 may measure a first height level h1 when the user raises their hand upward and a second height level h2 when the user puts their hand downward. The hemodynamic parameter estimating circuit 1200 may determine an intermediate point between the first height level h1 and the second height level h2 as a reference level ‘hr’. The hemodynamic parameter estimating circuit 1200 may estimate the true heart level of the user from the reference level ‘hr’. The hemodynamic parameter estimating circuit 1200 may estimate the user's arm length from the difference between the reference level ‘hr ’ and the first height level hl or the difference between the reference level ‘hr’ and the second height level h2. The hemodynamic parameter estimating circuit 1200 may use the user's arm length when estimating the blood vessel length.

Referring to FIGS. 2, 3, and 5B, the hemodynamic parameter estimating circuit 1200 may determine the reference level ‘hr’ from the trajectory of the user's hand. For example, the hemodynamic parameter estimating circuit 1200 may determine the reference level ‘hr’ based on a point perpendicular to the ground in the trajectory of the user's hand. The hemodynamic parameter estimating circuit 1200 may estimate the true heart level of the user from the reference level ‘hr’. The hemodynamic parameter estimating circuit 1200 may estimate the arm length of the user from the radius of curvature of the trajectory of the user's hand. The hemodynamic parameter estimating circuit 1200 may use the user's arm length when estimating the blood vessel length.

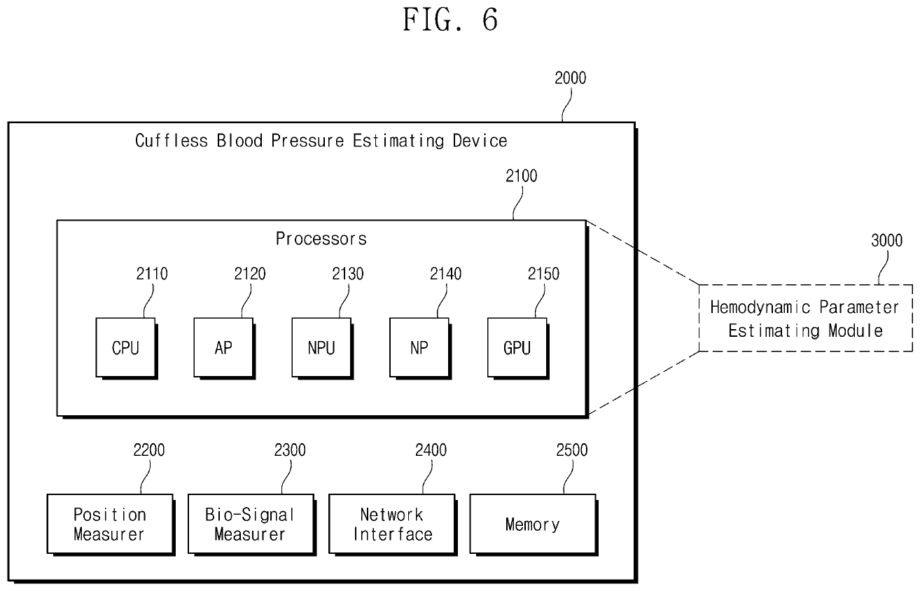

FIG. 6 is a block diagram illustrating a cuffless blood pressure estimating device 2000, according to an embodiment of the present disclosure. Referring to FIGS. 2, 3, and 6, the cuffless blood pressure estimating device 2000 may include a hemodynamic parameter estimating module 3000, processors 2100, a position measurer 2200, a bio-signal measurer 2300, a network. Interface 2400, and a memory 2500.

At least one of the processors 2100 may execute the hemodynamic parameter estimating module 3000. When the hemodynamic parameter estimating module 3000 is executed, at least one of the processors 2100 may estimate the hemodynamic parameters from height levels and user's bio-signals. For example, when the hemodynamic parameter estimating module 3000 is executed, at least one of the processors 2100 may estimate the hemodynamic parameters from height levels and user's bio-signals based on the machine learning algorithm or the state estimation algorithm with the hemodynamic state space model. When the hemodynamic parameter estimating module 3000 is executed, at least one of the processors 2100 may extract information about the blood pressure from the hemodynamic parameters.

Except for the processors 2100 executing the hemodynamic parameter estimating module 3000, the cuffless blood pressure estimating device 2000 has the same structure as the cuffless blood pressure estimating device 1000 of FIG. 2 and performs the same operation. Thus, additional description will be omitted to avoid redundancy.

In the above embodiments, components according to the present disclosure are described by using the terms “first”, “second”, “third”, and the like. However, the terms “first”, “second”, “third”, and the like may be used to distinguish components from each other and do not limit the present disclosure. For example, the terms “first”, “second”, “third”, and the like do not involve an order or a numerical meaning of any form.

According to an embodiment of the present disclosure, a blood pressure may be estimated with a calibration-free function or a minimum calibration function through changes in the user's bio-signals with respect to the height level of the measurement site (e.g., the user's finger or wrist). In addition, according to an embodiment of the present disclosure, simultaneously with blood pressure being estimated, other hemodynamic information such as blood density and blood vessel elasticity may also be estimated.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. Accordingly, the scope of the present disclosure should not be limited to the above-described embodiments and should be defined by equivalents of the claims as well as the claims to be described later.

What is claimed is:

1. A cuffless blood pressure estimating device comprising:
a hemodynamic parameter estimating circuit configured to measure at least two height levels based on position information output from at least one position detection sensor, to measure user's bio-signals respectively at the height levels, and to estimate a blood pressure from the height levels and the user's bio-signals based on a machine learning algorithm or a state estimation algorithm with a hemodynamic state space model; and
at least one processor configured to control the hemodynamic parameter estimating circuit.

2. The cuffless blood pressure estimating device of claim 1, wherein the hemodynamic parameter estimating circuit is configured to determine a reference level that represents a heart height level information of a user based on the height levels.

3. The cuffless blood pressure estimating device of claim 2, wherein the hemodynamic parameter estimating circuit is configured to estimate the blood pressure from a first difference value, which is a difference between the reference level and the first height level, and a second difference value, which is a difference between the reference level and the second height level.

4. The cuffless blood pressure estimating device of claim 1, wherein the hemodynamic parameter estimating circuit includes:
a position measurer including the at least one position detection sensor and configured to measure the height levels;
a bio-signal measurer configured to measure the user's bio-signals; and
a hemodynamic parameter estimator configured to estimate the blood pressure from the height levels and the user's bio-signals based on the machine learning algorithm or the state estimation algorithm with the hemodynamic state space model.

5. The cuffless blood pressure estimating device of claim 4, wherein the at least one position detection sensor includes at least one of an accelerometer, a gyroscope, a magnetometer, a barometer, an altimeter, a variometer and a distance measurement sensor.

6. The cuffless blood pressure estimating device of claim 5, wherein each of the user's bio-signals includes a photoplethysmogram (PPG) signal, and
wherein the bio-signal measurer includes a PPG sensor.

7. The cuffless blood pressure estimating device of claim 6, wherein the hemodynamic parameter estimating circuit is configured to:
estimate hemodynamic parameters from the height levels and the user's bio-signals based on the machine learning algorithm or the state estimation algorithm with the hemodynamic state space model; and
extract information about the blood pressure from the hemodynamic parameters to estimate the blood pressure.

8. The cuffless blood pressure estimating device of claim 7, wherein the hemodynamic parameter estimating circuit is configured to:
extract a plurality of features from each of the user's bio signals; and
estimate the hemodynamic parameters from the height levels and the plurality of the features based on the machine learning algorithm or the state estimation algorithm with the hemodynamic state space model.

9. The cuffless blood pressure estimating device of claim 6, wherein each of the user's bio-signals further includes an electrocardiogram (ECG) signal, and
wherein the bio-signal measurer further includes an ECG sensor.

10. The cuffless blood pressure estimating device of claim 7, wherein the hemodynamic parameters include information on one or more of the blood pressure, a vessel density, a vessel elastic modulus, a vessel wall thickness, a vessel radius, and/or a vessel length.

11. The cuffless blood pressure estimating device of claim 7,
wherein the hemodynamic parameter estimator is configured to estimate the hemodynamic parameters based on Equations 1 to 5 below,
X(m)=[Pa, ρ, E, T(m), L]^{T} [Equation 1]
X(m)=f(X(m−1), H(m)) [Equation 2]
Z(m)=[PPG(m), PTT(m)]^{T} [Equation 3]
PPG(m)=g1(X(m), H(m)) [Equation 4]
PTT(m)=g2(X(m), H(m)) [Equation 5]
where, ‘m’ represents an index of height level of the position detection sensor, ‘k’ represents the number of measured features, X(m) represents a hemodynamic parameter vector, Z(m) represents a feature vector having a size of k×1, Pa represents a blood pressure, ρ represents a blood density, ‘E’ represents an elastic modulus of blood vessels, T(m) represents a thickness of a vessel wall, R(m) represents a vessel radius, ‘L’ represents a vessel length, H(m) represents a difference value between a reference level and the m-th height level of the position detection sensor, PPG(m) represents a feature vector of a PPG signal measured at the m-th height level of the position detection sensor, PTT(m) represents a PTT value measured at the m-th height level of the position detection sensor, and f( ) and g( )represent functions based on hemodynamic formulas or mathematical models trained by a machine learning algorithm.

12. A method of operating a cuffless blood pressure estimating device for estimating a blood pressure, the method comprising:
measuring at least two height levels based on position information output from at least one position detection sensor;
measuring user's bio-signals respectively at the height levels; and
estimating a blood pressure from the height levels and the user's bio-signals based on a machine learning algorithm or a state estimation algorithm with a hemodynamic state space

13. The method of claim 12, further comprising:
determining a reference level that represents a heart height level information of a user based on the height levels.

14. The method of claim 13, wherein the estimating the blood pressure from the height levels and the user's bio-signals based on the machine learning algorithm or the state estimation algorithm with the hemodynamic state space model includes estimating the blood pressure from a first difference value, which is a difference between the reference level and the first height level, and a second difference value, which is a difference between the reference level and the second height level.

15. The method of claim 14, wherein the estimating of the blood pressure from the first difference value, which is the difference between the reference level and the first height level, and the second difference value, which is the difference between the reference level and the second height level includes determining the reference level from the first height level and the second height level.

16. The method of claim 15, wherein each of the user's bio-signals includes a photoplethysmogram (PPG) signal.

17. The method of claim 16, wherein the estimating the blood pressure from the height levels and the user's bio-signals based on the machine learning algorithm or the state estimation algorithm with the hemodynamic state space model includes:
estimating hemodynamic parameters from the height levels and the user's bio-signals based on the machine learning algorithm or the state estimation algorithm with the hemodynamic state space model; and
extracting information about the blood pressure from the hemodynamic parameters.

18. The method of claim 17, wherein the estimating hemodynamic parameters includes:
extracting a plurality of features from the each of the user's bio signals; and
estimating the hemodynamic parameters from the height levels and the plurality of the features based on the machine learning algorithm or the state estimation algorithm with the hemodynamic state space model.
